(19) United States
(12) Patent Application Publication
(10) Pub. No.: US 2010/0261177 A1
(43) Pub. Date: Oct. 14, 2010

(54) INCREASING SPECIFICITY IN A scFV SCREEN USING DUAL BAIT REPORTERS

(75) Inventors: Michael P. Weiner, Guilford, CT (US); Michael I. Sherman, Glen Ridge, NJ (US)

Correspondence Address: BANNER & WITCOFF, LTD., 1100 13th STREET, N.W., SUITE 1200, WASHINGTON, DC, 20005-4051, US

(73) Assignee: AFFOMIX CORPORATION, New Haven, CT (US)

(21) Appl. No.: 12/676,053

(22) PCT Filed: Sep. 17, 2008
(86) PCT No.: PCT/US08/76662

§ 371 Date: Jun. 8, 2010

Related U.S. Application Data
(60) Provisional application No. 60/972,842, filed on Sep. 17, 2007.

(51) Int. Cl. C12Q 1/68; C12N 1/21; C12N 5/10; C12N 1/19

(52) U.S. Cl. 435/6; 435/252.3; 435/325; 435/254.2; 435/348

(57) ABSTRACT

To increase the efficiency of the selection of antibodies of desired specificity, we create multi-bait strain(s) in which one bait is the target and one or more bait(s) are non-target. The non-target bait(s) may use one or more DNA-binding domain(s) that differ(s) from that of the target bait and thereby activate one or more different reporters from that activated by the target bait. Library hits that activate both sets of reporters are presumed to be inadequately specific and can be eliminated from further consideration. Alternatively, a non-target bait may be replaced with a second target bait, and hits selected that activate both sets of reporters. Other combinations of elements can be used.

### TECHNICAL FIELD OF THE INVENTION

This invention is related to the area of antibodies. In particular, it relates to methods for obtaining desirable antibodies.

### BACKGROUND OF THE INVENTION

The term “proteomics” has been applied to efforts to describe parallel processing systems that permit analysis of most or all proteins encoded by an organism. Such analyses are highly informative in the determination of the causes of normal or aberrant cellular behavior. Proteins can modulate cellular behavior either through mutation, over- or under-expression or and/or posttranslational modification. Antibodies can greatly facilitate analyses of protein integrity, quantity and modification.

Immunoglobulin G (IgG) is a prototypic antibody (Ab) molecule (FIG. 1). IgG consists of two light and two heavy polypeptide chains connected within a hinge region by disulfide bonds. The portion of the Ab that binds its antigen (Ag) is also known as the “Fab region”, or the “combining site”; the Fc portion of IgG serves as the recognition site for components of the immune system (complement and phagocytic cells). The sequence of the Fab region is much more variable than the rest of the Ab molecule, particularly the so-called “hypervariable region”, which determines the specificity of the antibody for binding to its cognate Ag.

X-Ray crystallography studies of Ag-Ab interactions show that the antigenic determinant nestles in a cleft formed by the combining site of the Ab. Thus, one concept of Ag-Ab interactions is that of a key (the Ag) which fits into a lock (the Ab). The bonds that hold the Ag to the Ab combining site are all non-covalent in nature. These include hydrogen bonds, electrostatic bonds, Van der Waals forces and hydrophobic bonds. Multiple bonding between the Ag and the Ab ensures that the Ag will be bound tightly to the antibody. Since Ag-Ab reactions occur via non-covalent bonds, they are by their nature reversible.

Ab affinity is the strength of the reaction between an antigenic determinant and a combining site on the Ab. In other words, affinity is the net result of the attractive and repulsive forces operating between the antigenic determinant and the combining site of the Ab. Affinity is characterized by an equilibrium constant that describes the Ag-Ab reaction as illustrated in the formula: Ag+Ab [symbol] AgAb. Applying the Law of Mass Action: K_{eq}=[AgAb]/{[Ag][Ab]} and K dissociation={[Ag][Ab]}/[AgAb]. Most Abs have a high affinity for their Ags.

Whereas affinity refers to the strength of binding between a single antigenic determinant and a single Ab combining site, avidity refers to the overall strength of binding between multivalent Ags and Abs. Avidity is more than the sum of the individual affinities.

Specificity is inversely related to the number of different antigenic determinants to which an individual Ab combining site will interact or to the number of different Ags with which an individual Ab or population of Ab molecules (in the case, e.g., of polyclonal antibodies) will react: the smaller the number of different antigenic determinants or Ags that will interact with an Ab, the greater the specificity of that Ab. In general, it is desirable to have a high degree of specificity in Ag-Ab reactions. Abs can distinguish differences in 1) the primary structure of an Ag, 2) isomeric forms of an Ag, and 3) secondary and tertiary structure of an Ag.

Cross reactivity refers to the ability of an individual Ab combining site to react with more than one antigenic determinant or the ability of a population of Ab molecules to react with more than one Ag. Cross reactions arise because the cross-reacting Ag shares an “epitope” in common with the immunizing Ag or because it has an epitope that is structurally similar to one on the immunizing Ag (multispecificity).

Approaches to Ab selection typically involve a multi-step process; initial efforts are made to identify Abs that bind to target Ag with at least a minimum level of affinity (or avidity). Only once such criteria have been satisfied are the Abs further evaluated for specificity, i.e., lack or cross-reactivity with non-target Ags. For some Ags it has been notoriously difficult to identify Abs of adequate specificity for a particular purpose. The sequential process of selection on the basis of affinity followed by the subsequent elimination of Abs that demonstrate unacceptable levels of cross reactivity is tedious, time consuming and expensive, particularly if the target Ag is expensive and/or difficult to produce.

Although the state of the art of generating antibodies against a single protein Ag has reached sophisticated levels, efforts to generate antibodies against the very large number of proteins that would be required for proteomic analysis have been stymied by problems of cost and scale. The generation of Abs against a protein Ag target typically involves the costly and time-consuming process of Ag purification and subsequent generation in animals of either poly- or monoclonal Ab and selection of those Abs that satisfy some minimum criteria for affinity and specificity. Ab generation in animals takes several weeks. A rate-limiting consideration of these conventional methods has often been the milligram amounts of purified protein needed, not only for injection into animals to generate Abs but also for isolation and characterization of Abs possessing suitable affinities and specificities. Phage display, an in vitro alternative for Ab selection with certain advantages over animal immunization, still suffers from the requirement for large amounts of purified Ag.

The need for milligram amounts of Ag for selection of Abs either in vivo or in vitro is not only an issue of cost; only 60-70% of all proteins can be readily purified and many of the purification procedures for proteins that are amenable to purification are labor-intensive and protracted. In particular, membrane-associated proteins, proteins containing certain cofactors and those requiring complexes for stability are very difficult to express and purify for subsequent use in Ab production. The use of intracellular expression of potential Ags, including both full-length and partial open reading frames, is one way to eliminate the need for preparing and purifying proteins.

The yeast two-hybrid (Y2H) assay can be used as a means for identifying Abs that bind with high affinities to Ags. Intracellular expression of the Ag eliminates the need for protein purification.

Y2H has been utilized extensively for identifying protein-protein interactions due to the ease of screening large numbers of potential interactors (Fields 1989; Chien 1991). In the interaction trap version of the Y2H system (Gyuris, 1993), a known protein, usually referred to as the “bait,” is fused to the carboxyl-terminus of the bacterial LexA protein containing the LexA operator-DNA binding domain (DBD). The cognate DNA binding element of the lexA operator is incorporated upstream of both a selectable auxotrophic reporter gene (typically Leu or His) integrated into the yeast genome and the lacZ gene on an autonomously replicating plasmid. Genes encoding target proteins, usually referred to as “prey,” are cloned as either random sequences or cDNAs fused to the carboxyl-terminus of a transcription activation domain (AD). Association of an AD-prey fusion with the DBD-bait results in reconstitution of a functional transcription factor and expression of the auxotrophic and lacZ reporter genes (FIG. 2). In this way Y2H is used to detect and define contacting proteins or protein domains. However, because it is necessary to retransform and select for an entire cDNA library for each protein studied, this approach has not been suitable for large-scale interaction mapping. Subsequently, interaction mating was developed as a means to re-use libraries (Bendixen 1994) and to assess interactions for larger, related collections of proteins (Finley 1994), proteomes (Bartel 1996; Giot 2003; Ito 2000; Li 2004; Uetz 2000) or members of individual biophysical complexes (Fromont-Racine 1997).

Abs can function in yeast. Several groups have developed in vivo assays for functional intracellular Abs using a two-hybrid approach in either yeast or mammalian cells (e.g., Portner-Taliana 2000; der Maur 2002). Typically, a single chain variable region of an Ab (“scFv”; see FIG. 1) that is linked to a transcriptional transactivation domain interacts with a target Ag linked to a DNA binding domain, thereby activating a reporter gene (FIG. 2). Several Abs have been characterized as being able to bind their target Ag in this two-hybrid format. When such Abs have been produced and tested in vitro, they have demonstrated the ability to bind their cognate Ag.

There is a continuing need in the art to generate efficiently Abs of desired specificity to most or all proteins encoded by an organism.

### SUMMARY OF THE INVENTION

According to one aspect of the invention, a method is provided for detecting an interaction between a polypeptide Ag and a single chain antibody variable fragment (scFv). A population of host cells is cultured. The host cells contain:
a first reporter gene which expresses a first reporter protein when the first reporter gene is activated by a polypeptide which includes a transcriptional activation domain when the transcriptional activation domain is brought into sufficient proximity to a first binding site on or adjacent to the first reporter gene;
a second reporter gene which expresses a second reporter protein when the second reporter gene is activated by a polypeptide which includes a transcriptional activation domain when the transcriptional activation domain is brought into sufficient proximity to a second binding site on or adjacent to the second reporter gene;
a first hybrid protein comprising:
   a first DNA-binding domain that recognizes the first binding site on or adjacent to the first reporter gene;
   a first polypeptide antigen;
a second hybrid protein comprising:
   the transcriptional activation domain;
   a single chain antibody variable fragment;
a third hybrid protein comprising:
   a second DNA-binding domain that recognizes the second binding site on or adjacent to a second reporter gene;
   a second polypeptide antigen.

Interaction between the first polypeptide antigen and the single chain antibody variable fragment in the host cell causes the transcription activation domain to activate transcription of the first reporter gene. Interaction between the second polypeptide antigen and the single chain antibody variable fragment in the host cell causes the transcription activation domain to activate transcription of the second reporter gene. The cells in the population are homogeneous with respect to the polypeptide antigen and heterogeneous with respect to the single chain antibody variable fragment. Expression of the first reporter gene is indicative of an interaction between the first polypeptide antigen and the single chain antibody variable fragment in a cell and expression of the second reporter gene is indicative of an interaction between the second polypeptide antigen and the single chain antibody variable fragment in the cell. Cells from among the population are selected. The selected cells (i) express the first reporter gene but not the second reporter gene, or (ii) express the first and the second reporter genes.

According to another aspect of the invention, another method is provided for detecting an interaction between a polypeptide antigen and a single chain antibody variable fragment. A population of first host cells is cultured. The first host cells contain:
a first reporter gene which expresses a first reporter protein when the first reporter gene is activated by a polypeptide which includes a transcriptional activation domain when the transcriptional activation domain is brought into sufficient proximity to a binding site on or adjacent to the first reporter gene;
a first hybrid protein comprising:
   a first DNA-binding domain that recognizes the binding site on or adjacent to the first reporter gene;
   a first polypeptide antigen;
a second hybrid protein comprising:
   the transcriptional activation domain;
   a single chain antibody variable fragment;
      wherein cells in the population of first host cells are homogeneous with respect to the first polypeptide antigen and heterogeneous with respect to the single chain antibody variable fragment.

A DNA molecule encoding the second hybrid protein is transferred from a cell which expresses the first reporter gene to a second host cell. The second host cell comprises:
a second reporter gene which expresses a second reporter protein when the second reporter gene is activated by a polypeptide which includes a transcriptional activation domain when the transcriptional activation domain is brought into sufficient proximity to a second binding site on or adjacent to the second reporter gene;
a third hybrid protein comprising:
   a second DNA-binding domain that recognizes the second binding site on or adjacent to a second reporter gene;
   a second polypeptide antigen.

Interaction between the first polypeptide antigen and the single chain antibody variable fragment in the first host cell causes the first transcription activation domain to activate transcription of the first reporter gene. A second host cell is selected. Interaction in the second host cell between a scFv selected in the first host cell and the second polypeptide antigen causes the second transcription activation domain to activate transcription of the second reporter gene. The second host cell comprises the DNA molecule encoding the second hybrid protein but (i) does not express the second reporter gene in the second host cell, or (ii) does express the second reporter gene in the second host cell. Expression of the first reporter gene in a cell in the first population of host cells is indicative of an interaction between the first polypeptide antigen and the single chain antibody variable fragment. Expression of the second reporter gene is indicative of an interaction between the second polypeptide antigen and the single chain antibody variable fragment in the second host cell.

According to still another aspect of the invention, a population of host cells is provided for use in selecting single chain variable regions. The cells comprise:
a first reporter gene which expresses a first reporter protein when the first reporter gene is activated by a polypeptide which includes a transcriptional activation domain when the transcriptional activation domain is brought into sufficient proximity to a first binding site on or adjacent to the first reporter gene;
a second reporter gene which expresses a second reporter protein when the second reporter gene is activated by a polypeptide which includes a transcriptional activation domain when the transcriptional activation domain is brought into sufficient proximity to a second binding site on or adjacent to the second reporter gene;
a first hybrid protein comprising:
   a first DNA-binding domain that recognizes the first binding site on or adjacent to the first reporter gene;
   a first polypeptide antigen;
a second hybrid protein comprising:
   the transcriptional activation domain;
   a single chain antibody variable fragment;
a third hybrid protein comprising:
   a second DNA-binding domain that recognizes a second binding site on or adjacent to a second reporter gene;
   a second polypeptide antigen.
      wherein cells in the population are homogeneous with respect to the polypeptide antigen and heterogeneous with respect to the scFv.

Interaction between the first polypeptide antigen and the single chain antibody variable fragment in the host cell causes the transcription activation domain to activate transcription of the first reporter gene. Interaction between the second polypeptide antigen and the single chain antibody variable fragment in the host cell causes the transcription activation domain to activate transcription of the second reporter gene.

According to yet another aspect of the invention, a kit is provided for interaction screening of polypeptides. The components of the kit are in a single or divided container. The components are:
a first reporter gene which expresses a first reporter protein when the first reporter gene is activated by a polypeptide which includes a transcriptional activation domain when the transcriptional activation domain is brought into sufficient proximity to a first binding site on or adjacent to the first reporter gene;
a second reporter gene which expresses a second reporter protein when the second reporter gene is activated by a polypeptide which includes a transcriptional activation domain when the transcriptional activation domain is brought into sufficient proximity to a second binding site on or adjacent to the second reporter gene;
a vector for making first hybrid protein comprising:
   a first DNA-binding domain that recognizes the first binding site on or adjacent to the first reporter gene;
   and an insertion site for sequence encoding a first polypeptide antigen;
a library of vectors encoding second hybrid proteins each of which comprises:
   the transcriptional activation domain;
   a single chain antibody variable fragment;
a vector for making a third hybrid protein comprising:
   a second DNA-binding domain that recognizes a second binding site on or adjacent to a second reporter gene;
   an insertion site for a sequence encoding a second polypeptide antigen;

Interaction between the first polypeptide antigen and a single chain antibody variable fragment in a host cell causes the transcription activation domain to activate transcription of the first reporter gene. Interaction between the second polypeptide antigen and a single chain antibody variable fragment in a host cell causes the transcription activation domain to activate transcription of the second reporter gene.

These and other embodiments which will be apparent to those of skill in the art upon reading the specification provide the art with means for efficiently generating and selecting and screening antibodies of desired specificity.

### BRIEF DESCRIPTION OF THE DRAWINGS

FIG. 1 Structure of immunoglobulin G (“IgG”). The structure consists of two heavy chains and two light chains. The predominant Ag-binding portion of IgG is within the so-called variable portion (“Fv”) of the Fab fragments, each of which consists of part of a heavy chain and part of a light chain. A recombinant form of a Fv region can be produced by linking the Fv component of a light chain with the Fv component of a heavy chain to produce a single chain variable fragment (“scFv”)

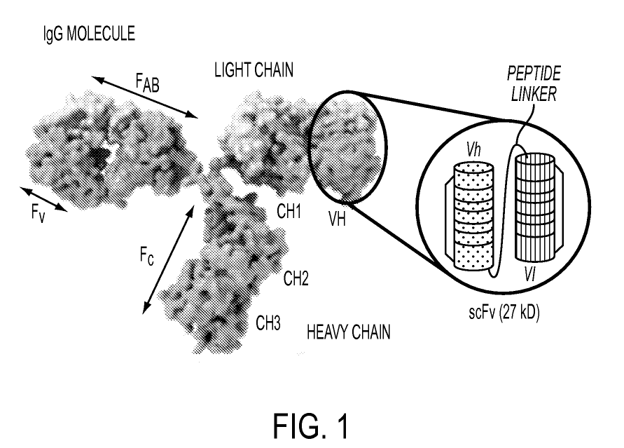

FIG. 2 Two-hybrid assay and prey cDNA vectors. (A) The two-hybrid assay, which permits analysis of proteins in an intracellular setting, requires the expression of two fusion proteins. Individually, neither the DNA binding domain (DBD) nor activation domain (AD) can activate transcription in yeast. The prey library consists of scFv cDNAs.

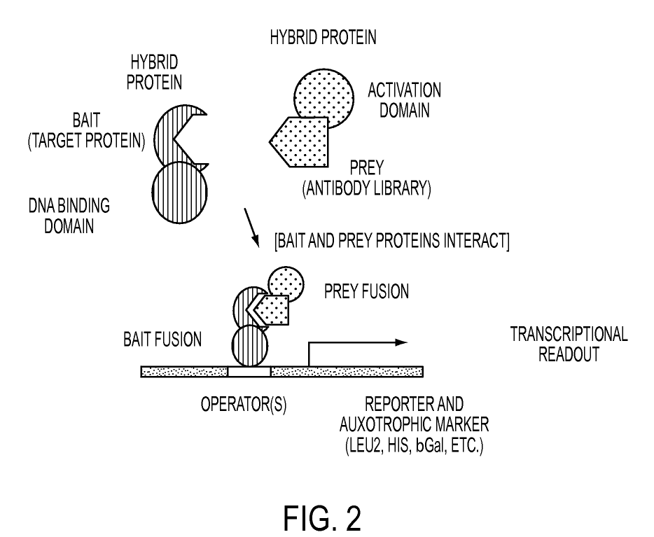

FIG. 3 Method for library construction. Seven DNA fragments corresponding to the 7 framework portions of an intracellularly-functional scFv were synthesized using overlapping oligonucleotides and cloned into E. coli vectors. DNAs corresponding to the 6 CDRs (3 each in the heavy and light genes) were amplified using mutagenic primers and spliced between the framework portions of the final scFv. In the second step, the VH and VL genes were each separately cloned into different vectors. The two gene fragments were then finally cloned into the yeast two-hybrid vector and transformed into the appropriate yeast strain.

FIG. 4 Re-test of bait specificity. ScFvs were rescued in E. coli and then re-introduced into the appropriate yeast strain. These Ab clones were then re-tested against the other 17 remaining baits. Shown in this example are tests for 5 scFvs against 8 baits. Co-selection plates indicate that both prey and bait vectors are present in the diploid yeast. Interaction plates contain His-minus minimal medium. Target baits on agar plates, (clockwise from top right); Akt 1-481, Raf 542-647, Bcl2 1-234, Src 150-249, p53 160-292, p53 95-292, Raf 191-271, Akt 79-144.

FIG. 5 Dual bait concept. The target and irrelevant bait(s) are made hybrid with DNA binding domains that each recognize a different DNA binding site. An Ab that recognizes both target bait and non-target bait is deemed undesired and selected against on a plate containing an appropriate amount of 5-FOA.

FIG. 6 Dual-bait selection to eliminate Abs that bind to the DNA Binding Domain (DBD) hybrid to the target bait. In the non-limiting example illustrated, the bait used for counter-selection is a mutated DBD (“DBD1*”) that is a mutated version of DBD1 (the DBD hybrid with the target bait) such that the mutation extinguishes the ability of the DBD to bind to its cognate DNA-binding sequence.

FIG. 7. The use of dual baits to enhance selection.

### DETAILED DESCRIPTION OF THE INVENTION

In this description yeast systems are often mentioned. These systems are interchangeable with mammalian, insect, and bacterial cells for intracellular interaction screening. Thus when yeast two-hybrid (Y2H) systems are mentioned, these are representative of any intracellular two-hybrid screening system. See for example Fiebitz, “High-throughput mammalian two-hybrid screening for protein-protein interactions using transfected cell arrays” BMC Genomics. 2008; 9: 68; Institute Pasteur, WO/2001/073108 “A Bacterial Two-Hybrid System For Protein Interaction Screening.”

When antibodies (Abs) or monoclonal antibodies (mAbs) are mentioned, these in the first instance refer to single chain variable chain molecules (scFv) which can be expressed in cellular screening systems. The scFv molecules can be routinely converted into full Ab molecules which share the same CDR regions. Ags are typically amino acid oligomers or polymers of about 4 to about 5000 residues in length. These are variously referred to as polypeptides or proteins or epitopes.

An integrated approach to generation and selection of scFv molecules is designed to facilitate the identification of individual scFv molecules. In addition it permits generation of a very large populations of scFv molecules, each of which has been selected for ability to bind with high affinity and selectivity to its target Ag (Ag). The inventors have developed means to improve the efficiency of selecting desirable Abs by the use of dual-bait strains and counter-selection.

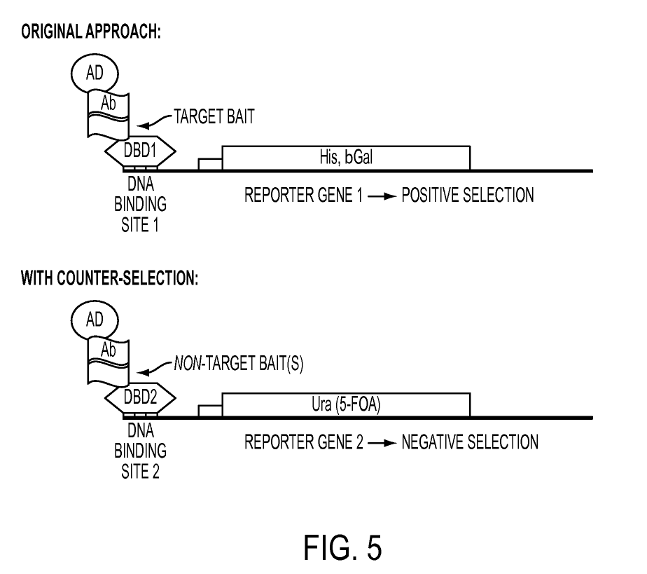

To increase the efficiency of the selection process we create multi-bait strain(s) in which one bait is the target and one or more bait(s) are non-target. The non-target bait(s) can use one or more DNA-binding domain(s) that differ(s) from that of the target bait and thereby activate one or more different reporters from that activated by the target bait. Library hits that activate both sets of reporters are presumed to be inadequately specific and can be eliminated from further consideration. This counter-selection approach is intended to reduce selection of interactors that are less specific than desired. One non-limiting example of counter-selection uses a Ura+ reporter and 5-FOA [5-fluoroorotic acid] to suppress growth of scFv library members that activate the counter-selected gene (URA) (FIG. 5).

In one non-limiting example, multiple polypeptide antigens are fused in a hybrid with a DBD. The multiple polypeptides may be related or unrelated. The multiple polypeptides form a linear array of antigens or epitopes.

Selection of cells can be performed using any technique known in the art. These may include antibiotic selections, antimetabolite selections, toxin selections, etc. Screens for conditional growth can be used using different nutrients, different temperatures, etc.

The counter-selection can occur simultaneously within the same yeast cell in which the original bait is screened, in which case the dual baits may each control different DNA binding sites such that the target bait is attached to a DNA binding domain (DBD) that recognizes a DNA sequence upstream of one or more selectable or detectable markers (for example βGal, His, Leu, etc.) and the counter-selected bait is attached to a DBD that recognizes a different DNA binding site located upstream of a different marker, for example URA. One or more counter-selected baits can be made hybrid with the second DBD. In a non-limiting example, separate control of the expression of each bait can be used to individually test one bait at a time. The same DBD could be used for all baits. For example, the target bait is expressed and the non-target baits are repressed, and the library is selected on URA− plates. Then, the desired-bait expression is repressed and the non-target baits are induced for expression and the cells are plated onto plates containing 5-FOA.

In one non-limiting example, the counter selection can occur after the primary screen with the target bait hybrid. In this example, selection occurs first with the target bait, and then with one or more non-target baits on one or more counter-selected markers. Since the primary and secondary screens are not performed at the same time, it is possible to use the same DBD for both the desired and non-target baits. In a non-limiting example, the positive ‘hits’ from the screen with the target bait can be pooled and tested en masse in a strain encoding one or more non-target baits. It is to be understood that in most embodiments described here, non-target baits can be replaced with second target baits which can be used for enhanced selection methods.

The use of a conditionally essential reporter gene (such as an auxotrophic marker) in the two-hybrid interactor system permits the selection of scFv molecules because in the absence of expression of the conditionally essential reporter gene, the mated cells fail to proliferate in minimal medium. On the other hand, it is often desirable to differentiate qualitative as well as quantitative features regarding the scFv molecules that are not immediately obvious solely by virtue of survival of the cells under selective growth conditions. As noted above, two key criteria for determining the potential value of selected Abs are affinity and specificity for the cognate Ag. Golemis and der Maur and their colleagues have separately shown that β-Galactosidase (βGal) activity can be used as a read-out to estimate the binding affinity of interacting bait and prey proteins in a two-hybrid screen (Serebriiskii 1999, 2000, 2005; Moll 2001). Briefly, the interaction of the bait and prey proteins leads to the transcription and translation of a βGal gene, whose presence can be detected and quantitated by the addition of a suitable substrate for the enzyme. Golemis' results (see Estojak 1995) indicate that the strength of interaction as predicted from such ρGal measurements generally correlates with that determined from direct binding studies in vitro, at least permitting broad discrimination of high-, intermediate-, and low-affinity interactions using the βGal assay. Moreover, some of the βGal reporters showed thresholds of activation, such that weak interactions were generally not detected. The Kd values predicted in the Golemis study spanned the range of 10⁻⁴ to 10⁻¹⁵ M. Significant discrimination in the Y2H assay occurred in the range of 10⁻⁸ M: for example, βGal activity read-outs obtained from proteins that interact in vitro with a Kd of 1×10⁻⁸ M were more than 10-fold higher than those for proteins with a Kd of 2.7×10⁻⁷. The range studied by der Maur was less extensive (Kd between 10⁻⁹-10⁻¹⁰ M), but once again the general correlation with βGal activity was observed.

Any of a number of binding studies can be carried out to establish whether or not an Ag-Ab reaction has occurred. The complexes formed between Ag and Ab can be detected directly or indirectly. The ease with which one can detect Ag-Ab interactions will depend on a number of factors, including affinity (the higher the affinity of the Ab for the Ag, the more stable will be the interaction and thus the ease with which the interaction can be detected) and avidity (reactions between multivalent Ags and multivalent Abs are more stable and thus easier to detect). An obvious consideration in characterization of Abs is the requirement for producing, purifying and tracking the Ab. There are many techniques known in the art for optimizing each of these processes.

Ability to grow and enzyme activity can be used to identify host cells that contain Abs bound to target Ag. Growth and enzyme activity can be scored visually, and thus semi-quantitatively. More quantitative approaches are taken to evaluate growth of the host cells or enzyme activity, or both. As just one of many non-limiting examples, growth of cells in liquid can be measured by turbidometric means or growth of cells on semi-solid or solid medium is determined by measurement of clone size. As further non-limiting examples, enzyme activity can be quantitatively evaluated by devices routinely used to measure colorimetric or fluorimetric molecules. Standardization of the two-hybrid conditions is an optional approach to improving estimation of the extent of the affinity of scFv molecules for their target Ags in the two-hybrid procedure. As non-limiting examples, time of incubation of the yeast and time of assay for enzyme activity can be kept constant from sample to sample. As another non-limiting example, enzyme activity is normalized to the number of cells assayed. Any of these or other approaches well known in the art are used to provide further calibration and thus improve predictivity of level of affinity between Ab and target Ag in the two-hybrid procedure.

Estojak (1995) and others have shown that by varying the number of operators before the reporter gene in a Y2H experiment, enzyme activity can be titrated in the yeast. In an alternative embodiment of the present invention, host clones are obtained in which interactions between proteins of known affinity are detected based on βGal activity and a calibration curve is generated for the relationship between protein affinity and βGal activity. In another embodiment, the number of operators on the βGal gene is selected such that an affinity threshold cut-off is predicted, namely, if the scFv-Ag interaction has an affinity that is not lower than the threshold cut-off, βGal activity is either undetectable or below a chosen level scored as a positive result. In a further embodiment, the threshold cut-off is Kd<10⁻⁶. In a further embodiment, the threshold cut-off is Kd<10⁻¹⁰. In a preferred embodiment, the affinity threshold cut-off is Kd<10⁻⁸.

A large number of other enzyme activities known in the art can be used as a means to monitor a positive response. There are also a number of alternative embodiments in which measurement of βGal activity is performed. As one set of non-limiting examples, any of a number of different βGal substrates known in the art is used. As one such non-limiting example, Xgal can be used. Among other substrates used to measure βGal activity are the following: nonFDG, ONPG and, CPRG. In one non-limiting example, βGal is measured in solution using yeast cells permeabilized in any of a number of ways known in the art.

In one embodiment, the affinity of selected scFvs is determined by direct analysis of an isolated scFv and its target Ag. In this embodiment an scFv selected in a two-hybrid procedure, as well as its target Ag, are prepared by any of a number of methods well known in the art. The affinity of the scFv for the target Ag is then measured in any of a number of ways defined in the art. As one preferred but non-limiting example, surface plasmon resonance is measured by means of a Biacore instrument. In this non-limiting example, kinetics are determined for the ability of a purified scFv to interact with Ag bound to a substratum in the presence of increasing amounts of unbound Ag. In one embodiment of this non-limiting example, the scFv is diluted to approximately 5 nM and equilibrated overnight with different concentrations of the target Ag ranging from 0 to 15 nM. After equilibration, the Biacore instrument is used to measure binding kinetics. In another non-limiting example biotinylated target Ag is bound to a Biacore sensor chip previously coated to maximal density with Neutravidin and binding kinetics are carried out in Hepes buffer (20 mM Hepes, 150 mM NaCl, pH 7.4) in the presence of varying amounts of unbound target Ag.

As noted in Table I, baits in the two-hybrid system can autoactivate. It can be tedious and inefficient to determine in a stepwise fashion which bait appears to interact with a given prey and, subsequently, to determine whether the measured interaction is an artifact of auto-activation. By analogy, it is inefficient to first determine which Abs bind to a given Ag target, only to determine subsequently that a selected Ab is unacceptable because it also demonstrates cross-reactivity for non-target Ags. The following approach is utilized as a way to eliminate from further consideration both inappropriate Ag baits that cause auto-activation in the two-hybrid system being used as well as Abs that suffer from unacceptable levels of cross reactivity with non-target Ags. In one of several embodiments, the procedure is used to identify a mAb that preferentially binds to one member of a protein family but not to related members of the same protein family. Similarly, specificity for a particular allelic variant can be identified. The variant can be a polymorphism or a mutation, for example.

In the non-limiting examples that follow, the efficiency of selecting desirable scFvs is increased by reducing the likelihood that non-specific interacting scFvs are selected for further analysis. The procedure improves the selection efficiency via the initial elimination of non-specific scFv interactors from the library through use of dual-bait strains and counter-selection.

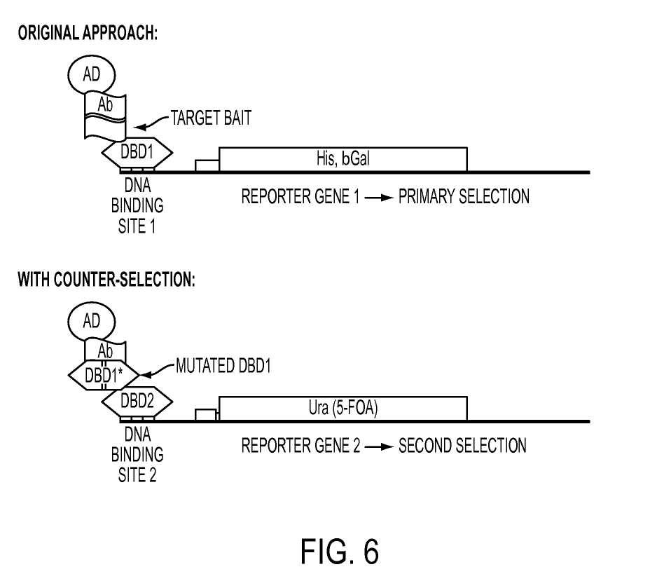

It is possible in two-hybrid selection to obtain false positive selections because the scFv is binding directly to the DBD rather than to the target bait, thereby triggering expression of the genes under control of the DNA-binding element. Accordingly, in another non-limiting example of the general dual-bait, counter-selection approach, one or more of the counter-selection baits is/are comprised of sequences similar to the DBD used for the selection but modified so as to not to allow the product to serve as a DNA-binding sequence (see, e.g., FIG. 6). Such inactivated DBDs are referred to in FIG. 6 and hereinafter as DBD*. The elimination of the DNA-binding capability of the DBD* can be achieved in any of a number of ways well known in the art, including point mutation, truncation and/or rearrangement. Suitable DBD*s for this purpose are readily identified by generating scFv molecules to a legitimate DBD and then testing such scFv molecules for binding to a candidate DBD*. A DBD* that is bound by one or more scFv molecules that bind to the legitimate DBD is useful as a counter-selection bait and can be used for this purpose as a hybrid with a legitimate DBD (see FIG. 6). In other non-limiting embodiments of this invention, one or more DBD* non-target baits is/are used in tandem or otherwise in combination with other non-target baits such that the counter-selection process is designed to eliminate in the same selection screen not only scFv molecules that bind to one or more non-targets similar to, but different from, the legitimate target, but also scFv molecules that bind to DBD* (and thus also to the corresponding DBD).

In the non-limiting examples presented hereinafter, a non-target bait can comprise DBD* or some other non-target sequence, and the baits can be used either separately or together, including in tandem.

In yet another non-limiting example of this general approach, bait expression is manipulated such that under a given set of circumstances (positive selection) Abs are selected that bind to the target bait hybrid with a DBD, whereas under an alternative set of circumstances (counter-selection) Abs are identified for elimination from further consideration if they bind to a non-target bait hybrid with a DBD. Only Abs that bind to a target bait under the set of circumstances used during positive selection are chosen for further consideration. Various parameters are used to differentiate conditions for positive selection versus conditions for negative selection. As a non-limiting example, the same Ab clone is tested in two separate yeast strains, one of which expresses a target bait hybrid to a DBD (strain A), the other of which expresses a non-target bait hybrid to the same DBD (strain B). Abs are selected for further consideration if they are scored positive only in strain A.

In yet another non-limiting example, counter-selection occurs after a primary screen to detect Abs that bind to the target bait. In this example, selection occurs first with the target bait and subsequently counter-selection occurs with one or more non-target baits. Since the positive selection and counter-selection are not performed at the same time, the same DNA-binding site is optionally used for both the target bait and the non-target bait. As one embodiment of this non-limiting example, yeast are exposed temporally to two different inducers such that a hybrid protein containing the target bait is produced in response to exposure to a first inducer whereas a hybrid protein containing a non-target bait is produced in response to exposure to a second inducer. Abs that bind to the first hybrid protein when it is produced but not to the second hybrid protein when it is subsequently produced are selected for further consideration.

In one non-limiting embodiment of a positive selection followed sequentially by a counter-selection, Abs selected for further consideration following the positive selection step are isolated and recloned en masse into a strain in which they are counter-selected by testing against one or more non-target baits.

In another non-limiting example, the counter-selection process precedes the positive selection. In this embodiment, cells containing Abs that bind to one or more non-target baits are selected against and thus only those cells containing Abs that have not detectably bound to the non-target bait(s) are further tested for binding to the legitimate target bait. Thus the initial counter-selection process removes from an Ab library those members that bind to particular non-target baits. In a non-limiting example, the target and non-target baits co-reside in the same host cells but expression of the non-target bait and the target bait are manipulated temporally by the use of different inducers and/or repressors.

In yet another non-limiting example, selection and counter-selection are carried out with a reporter that is utilized in selection in two different ways. As a non-limiting example, for positive selection, binding of an Ab to a target bait leads to expression of the Ura gene and allows yeast carrying such Abs to grow on Ura− plates. For counter-selection binding of an Ab to a non-target bait leads to expression of the Ura gene in the presence of 5-FOA, which leads to the failure of yeast carrying such Abs to grow. The positive selection and counter-selection phases are carried out temporally by the differential use of inducers and/or repressors or by transformation of the Ab-activation domain hybrid genes into different strains.

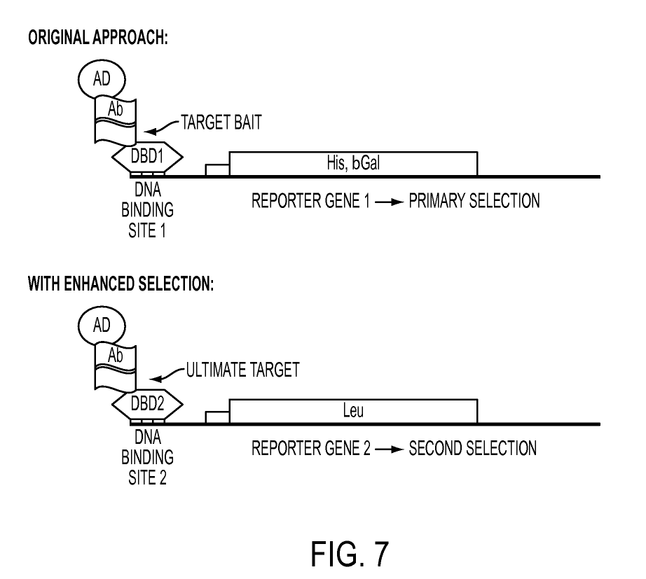

In a further embodiment, the dual baits are used to enhance the selection process. As a non-limiting example (FIG. 7), it may be desired to generate an Ab that is selected against a particular epitope of a protein but which can also bind to the full-length protein (it being the case that certain epitopes might be altered conformationally in the full-length protein or otherwise rendered inaccessible to an Ab selected for binding to the isolated epitope). Accordingly, the target bait, i.e., the epitope, is made part of a hybrid protein molecule with a first DBD (DBD1 in FIG. 7) and the full-length protein is made as part of a hybrid protein with a second different DBD (DBD2 in FIG. 7). An Ab must bind to both baits in order to be selected. This can be effected by having DBD1 bind to a cognate DNA-binding element of an operator incorporated upstream of a selectable auxotrophic reporter gene that activates a selectable auxotrophic agent (as a non-limiting example, a gene involving the Leu pathway) and by having DBD2 bind to a different cognate DNA-binding element of an operator incorporated upstream of a different selectable auxotrophic reporter gene that activates a different selectable auxotrophic agent (as a non-limiting example, a gene involving the His pathway) and the selection is then carried out in medium lacking both amino acids (in the examples given, Leu and His). The second selection is carried out simultaneously or sequentially.

It is sometimes difficult to express full-length proteins from higher organisms in yeast. Accordingly, in another embodiment of the enhanced selection process, a larger portion of the protein containing the target epitope, rather than the full sequence, is used in the second selection.

Other situations are contemplated in which enhanced selection is desirable. In one such other example, the objective is to identify Abs that bind to a multiplicity of different proteins that have common epitope sequences or conformations.

One should not a priori assume that a target protein or target epitope of a protein from a species other than the host cell when produced in the host cell will adopt the “native” conformation that it typically assumes in its native host cell. scFv molecules selected in two-hybrid against proteins not in their native conformation might be unsuitable as binders to their target proteins in vivo. This would, for example, diminish the value of such scFv molecules as therapeutic candidates. Additionally, some full-length proteins might fail to be produced or be poorly produced in yeast when compared to individual epitope; low levels of target protein production could reduce the likelihood of selecting desirable scFv molecules against that protein. Although in the case of non-yeast proteins, individual epitopes might be produced at higher levels than the full-length protein in yeast, the use of individual epitopes for selecting scFv molecules could preclude selection of scFv molecules that bind to discontinuous domains that are brought together by virtue of folding of the full-length protein. An approach to the above limitations is use of a scaffold or linker that creates a more “native” three-dimensional structure for a particular site within a protein that one desires to target. Such linkers or scaffolds could variously be comprised of amino acids, chemical entities or a combination of the two, although for in vivo applications, such as two-hybrid, the linkers and/or scaffolds are most conveniently comprised of amino acids. Protein modeling and/or three-dimensional determinations are typically used in the art to predict the optimal size and structure of the linkers and/or scaffolds.

In one embodiment of the foregoing invention, the target bait in a two-hybrid selection comprises one or more epitopes together with one or more linkers and/or scaffolds, in a hybrid protein with a DBD, in a way such that the epitope(s) assume(s) a more native structure. In another embodiment, a non-target bait that is comprised of the same one or more linkers and/or scaffolds [but not the target epitope(s)] in a hybrid with a second DBD is additionally present as a counter-selection such that selected scFv molecules are those that bind to the target epitope(s) rather than the linker(s) and/or scaffold(s).

Kits can be used to facilitate the use of the methods. The kits will typically comprises individual components in separate vessels all contained within one larger container. In some embodiments the DNA vectors and/or reporter genes are isolated. In some embodiments they may be in host cells. Cells may be provided in liquid, dehydrated, lyophilized, or frozen form. DNA may similarly be in liquid or dried form, isolated or in cells. It may be convenient to provide pre-made Ab libraries within the kit. Alternatively, consumers may prefer to make their own libraries. In the latter case, the kit may optionally comprise vectors comprising the framework for Ab complementarity determining regions (CDRs), without the CDRs. Optional reagents which may be supplied include enzymes for making the recombinant constructs, such as DNA ligase and restriction endonucleases, substrates for use in detecting reporter genes, cell growth media, selective agents for selection of cells which express a reporter gene. Written instructions may be supplied with the kit, related to the construction of the hybrid proteins and the assaying of reporter proteins. Written instructions may be supplied within the kit as a paper product, on a computer readable data storage medium, or as an address to an internet source.

It will be recognized that various recombinant techniques can be used to modulate the stringency required for reporter protein expression. This may involve use of one or a series of operator elements to which a positive or negative regulatory protein may bind. Spacing of such elements may be altered to alter stringency. Mutations may be used to alter stringency. Any techniques known in the art can be used to establish a robust assay that adequately distinguishes between signal and background.

As noted above, the methods of the invention can be used in a configuration in which the first and second reporters are expressed and assayed in one cell, or they can be expressed and assayed separately in more than one cell. First and second reporter can be expressed separately from a temporal perspective, by means of inducers or other conditional expression approaches.

Although a first and a second reporter protein are described throughout this description, additional reporter proteins can be used. These can be used as confirmation of the first and/or second reporter proteins. By using additional reporters, various levels of stringency of interaction can be assessed simultaneously. For example, one reporter can be non-stringent and the additional reporter can be more stringent. Alternatively, one reporter can be non-quantitative and the other may provide a quantitative read-out.

Although DNA binding domains are used throughout this description, RNA binding domains can also be used, as is known in the art. See, e.g., U.S. Pat. No. 5,750,667.

Transferring of genetic constructs from one cell to another can be done using any of the techniques known in the art. These include without limitation, mating, transformation, transfection, electroporation, cell fusion, coated-particle ballistic transfer, viral mediated transfer, liposome mediated transfer, nanoparticles mediated transfer, and mating. Transfers may be done using isolated (biochemically or genetically) genetic elements or using mixed populations of genetic elements, i.e., en masse.

Any type of reporter gene known in the art can be used, including enzymes, drug resistance proteins, fluorescent proteins, bioluminescent proteins. As mentioned above, in some situations quantitation of the reporter gene expression is desired. In others, quantitation may not be needed.

Culturing of host cells can be on liquid or solid medium. Mixed populations or homogeneous populations can be cultured. Conditions of nutrients, temperature, inducers, repressors, antibodies, etc. can be modified to manipulate expression of hybrids and reporter genes.

Populations of host cells may be homogeneous or heterogeneous. Homogeneous populations contain genetically identical cells. Heterogeneous populations contain cells which contain different genetic elements. The host cells in a population may be in any form, frozen, fresh, lyophilized, spores, etc. They may be in a culture or storage medium. They may be on a solid support. They may be contained within a vessel, compartment, or emulsion. They may be arrayed in microtiter dishes as a library. They may be supplied on an inoculating device. Similarly, vectors can be supplied in homogeneous or heterogeneous preparations in any physical form which permits the vectors to retain their biological activity and integrity.

Selection of cells can be accomplished by subjecting cells to non-permissive conditions in the absence of the desired genetic construct or desired protein-protein interaction. Selection can also be accomplished by identifying a desired phenotype that occurs only in the presence of the desired genetic construct or desired protein-protein interaction. Thus selected cells can be identified and non-selected cells may be deselected.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE

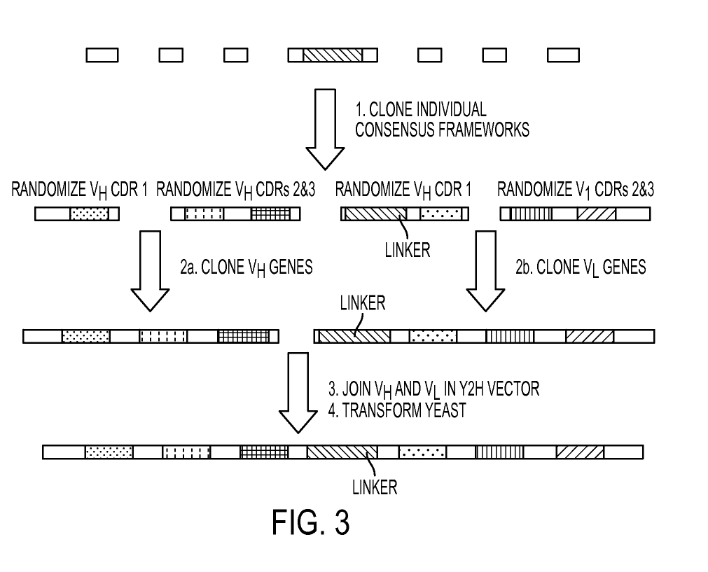

Specific Abs can be selected from a library pool. We generated a scFv library using a framework known to be functional in yeast and screened it with 24 baits. The choice of baits was relatively arbitrary and they are shown in Table I. The baits were of different sizes and constituted different portions of 7 different proteins. The constant framework template for the library was a consensus scFv. The CDRs of that scFv were randomized by PCR using mutagenic oligonucleotides (including oligonucleotides of different lengths to vary the size of some of the CDR regions) and then re-assembled into a full-length scFv (FIG. 3). Thus we made a defined scFv with a functional framework, then introduced variability into the CDRs of that construct by PCR.

TABLE 1

| Gene | Bait | | Length |
|---|---|---|---|
| Name | Name | Fragment | (aa) |
| AKT1 | 6 A9 | 360-408 | 48 |
| AKT1 | 6 B2 | 409-476 | 67 |
| AKT1 | 6 B5 | 79-144 | 65 |
| AKT1 | 6 B10 | 253-334 | 81 |
| AKT1 | 6 A2 | 1-108 | 108 |
| AKT1 | 6 C1 | 1-481 | 481 |
| ATF4 | 7 A11 | 1-352 | 352 |
| BCL2L | 6 G5 | 1-234 | 234 |
| RAF1 | 6 D6 | 56-131 | 95 |
| RAF1 | 6 D9 | 139-187 | 48 |
| RAF1 | 6 E1 | 29-131 | 102 |
| RAF1 | 6 E9 | 191-271 | 80 |
| RAF1 | 6 F5 | 422-536 | 114 |
| RAF1 | 6 F9 | 542-647 | 105 |
| RAF1 | 6 E5 | 349-609 | 260 |
| SRC | 6 G9 | 83-144 | 61 |
| SRC | 7 B9 | 150-249 | 99 |
| SYK | 7 C5 | 163-261 | 98 |
| SYK | 7 C10 | 370-613 | 243 |
| SYK | 7 A3 | 1-613 | 613 |
| TP53 | 7 G2 | 318-359 | 41 |
| TP53 | 7 F5 | 160-292 | 132 |
| TP53 | 7 F10 | 95-292 | 197 |
| TP53 | 7 G5 | 1-394 | 394 |

Table 1. Baits used in the described screen. Baits in bold font were found to be auto-activators and were not screened against the scFv library.

The 24 baits were screened against an scFv library using standard Y2H methods. The two phenotypic markers used in this experiment to indicate effective protein-protein interaction were the ability to grow in the absence of histidine (i.e., reversion to His prototrophy) and the gain of βGal activity. The selection was designed such that growth in the absence of His was “low-stringency” (although we also modulated the stringency of His selection with the inhibitor 3-AT; see below) whereas expression of βGal was “high stringency”; this allowed us to identify as many binders as possible in the initial selection and then to discriminate among them based on βGal activity).

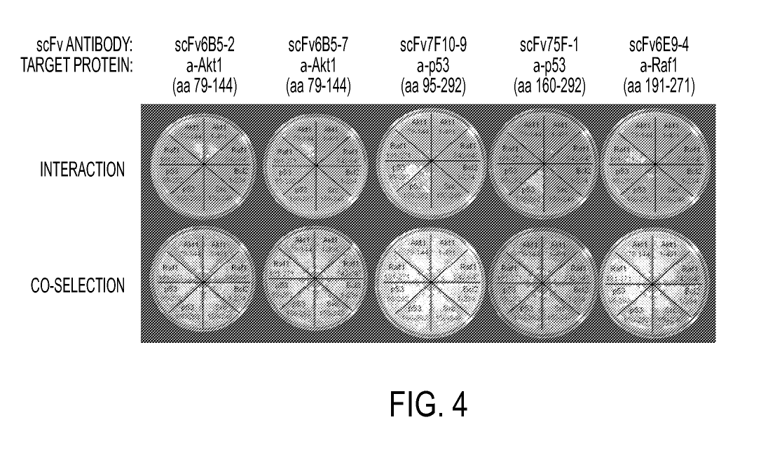

Six baits when cloned into the DBD vector were found to auto-activate (in bold font in Table 1). This is most likely due to the ability of these baits to recruit RNA polymerase to the DNA binding site without interaction through a prey. These baits were not pursued further. The 18 other baits were screened against the library using standard two-hybrid methods. Approximately 5-10 two-hybrid interaction clones with each bait (putative scFv ‘hits’) arising on His-minus minimal plates were then rescued into E. coli cells to isolate the scFv-containing prey vector. The plasmids were retransformed into yeast haploid cells and then further tested for i) interaction with the remaining 17 baits (as a test for specificity to the bait used in the original screen) and ii) βGal activity (see, e.g., FIG. 4). Several scFvs were found that reacted against several unrelated baits; these were considered non-specific and not pursued further.

Despite the small size of the scFv library, we isolated specific Abs against 4 different baits derived from 3 different proteins. It was very encouraging that several scFvs reacted against baits that included overlapping regions of the same protein (see for example 6B5-2, 6B5-3, and 7F10-9; see FIG. 4), suggesting that epitope recognition can be conserved in fragments of different sizes. FIG. 4 demonstrates further that whereas two of the Abs illustrated (scFv6B5-2, scFv75F-1) bound to the epitope against which they were selected but not to larger versions of the target Ag, another Ab (scFv6B5-7) bound both to the epitope used for selection and to the full-length target Ag.

### REFERENCES

The disclosure of each reference cited is expressly incorporated herein.
Bartel, P. L., Chien, C., Sternglanz, R. and Fields, S. (1993) In Hartley, D. A. (ed.), Cellular interaction in development: a practical approach. IRL Press, Oxford, pp. 153-179.
Bendixen C, Gangloff S, Rothstein R. A yeast mating-selection scheme for detection of protein-protein interactions. Nucleic Acids Res. 1994 May 11; 22(9):1778-9.
Buckholz R G, Simmons C A, Stuart J M, Weiner M P. Automation of yeast two-hybrid screening. J Mol Microbiol Biotechnol. 1999 August; 1(1):135-40.
Chien, C. T., Bartel, P. L., Sternglanz, R., and Fields, S. (1991) The two-hybrid system: a method to identify and clone genes for proteins that interact with a protein of interest. Proc. Natl. Acad. Sci. U.S.A. 88, 9578-9582.
der Maur A A, Zahnd C, Fischer F, Spinelli S, Honegger A, Cambillau C, Escher D, Pluckthun A, Barberis A. Direct in vivo screening of intrabody libraries constructed on a highly stable single-chain framework. J Biol. Chem. 2002 Nov. 22; 277(47):45075-85. Epub 2002 Sep. 4.
Estojak J, Brent R, Golemis E A. Correlation of two-hybrid affinity data with in vitro measurements. Mol Cell Biol. 1995 October; 15(10):5820-9.
Fiebitz, “High-throughput mammalian two-hybrid screening for protein-protein interactions using transfected cell arrays” BMC Genomics. 2008; 9: 68; Institute
Pasteur, WO/2001/073108 “A Bacterial Two-Hybrid System For Protein Interaction Screening.”
Fields, S, and Song, O. (1989) A novel genetic system to detect protein-protein interactions. Nature, 340, 245-246.
Finley R L Jr, Brent R. Interaction mating reveals binary and ternary connections between Drosophila cell cycle regulators. Proc Natl Acad Sci USA. 1994 Dec. 20; 91(26): 12980-4.
Fromont-Racine M, Rain J C, Legrain P. Toward a functional analysis of the yeast genome through exhaustive two-hybrid screens. Nat. Genet. 1997 July; 16(3):277-82.
Giot, L., Bader, J. S., Brouwer, C., Chaudhuri, A., Kuang, B., Li, Y., Hao, Y. L., Ooi, C. E., Godwin, B., Vitols, E., Vijayadamodar, G., Pochart, P., Machineni, H., Welsh, M., Kong, Y., Zerhusen, B., Malcolm, R., Varrone, Z., Collis, A., Minto, M., Burgess, S., McDaniel, L., Stimpson, E., Spriggs, F., Williams, J., Neurath, K., Ioime, N., Agee, M., Voss, E., Furtak, K., Renzulli, R., Aanensen, N., Carrolla, S., Bickelhaupt, E., Lazovatsky, Y., DaSilva, A., Zhong, J., Stanyon, C. A., Finley, R. L., Jr, White, K. P., Braverman, M., Jarvie, T., Gold, S., Leach, M., Knight, J., Shimkets, R. A., McKenna, M. P., Chant, J., and Rothberg, J. M. (2003) A protein interaction map of Drosophila melanogaster. Science 302, 1727-1736.
Gyuris J, Golemis E, Chertkov H, Brent R. Cdil, a human G1 and S phase protein phosphatase that associates with Cdk2. Cell. 1993 Nov. 19; 75(4):791-803.
Ito, T., Tashiro, K., Muta, S., Ozawa, R., Chiba, T., Nishizawa, M., Yamamoto, K., Kuhara, S., and Sakaki, Y. (2000) Toward a proteinprotein interaction map of the budding yeast: A comprehensive system to examine two-hybrid interactions in all possible combinations between the yeast proteins. Proc. Natl. Acad. Sci. U.S.A. 97, 1143-1147.
Li, S., Armstrong, C. M., Bertin, N., Ge, H., Milstein, S., Boxem, M., Vidalain, P. O., Han, J. D., Chesneau, A., Hao, T., Goldberg, D. S., Li, N., Martinez, M., Rual, J. F., Lamesch, P., Xu, L., Tewari, M., Wong, S. L., Zhang, L. V., Berriz, G. F., Jacotot, L., Vaglio, P., Reboul, J., Hirozane-Kishikawa, T., Li, Q., Gabel, H. W., Elewa, A., Baumgartner, B., Rose, D. J., Yu, H., Bosak, S., Sequerra, R., Fraser, A., Mango, S. E., Saxton, W. M., Strome, S., Van Den Heuvel, S., Piano, F., Vandenhaute, J., Sardet, C., Gerstein, M., Doucette-Stamm, L., Gunsalus, K. C., Harper, J. W., Cusick, M. E., Roth, F. P., Hill, D. E., and Vidal, M. (2004) A map of the interactome network of the metazoan C. elegans. Science 303, 540-543.
Moll, J. R., Ruvinov, S. B., Pastan, I., and Vinson, C. (2001) Designed heterodimerizing leucine zippers with a range of pls and stabilities up to 10̂15 M. Protein Sci. 10, 649-655.
Portner-Taliana A, Russell M, Froning K J, Budworth P R, Comiskey J D, Hoeffler J P. In vivo selection of single-chain antibodies using a yeast two-hybrid system. J Immunol Methods. 2000 Apr. 21; 238(1-2):161-72.
Serebriiskii, I., Khazak, V., and Golemis, E. A. (1999) A two-hybrid dual bait system to discriminate specificity of protein interactions. J. Biol. Chem. 274, 17080-17087
Uetz, P., Giot, L., Cagney, G., Mansfield, T. A., Judson, R. S., Knight, J. R., Lockshon, D., Narayan, V., Srinivasan, M., Pochart, P., Qureshi-Emili, A., Li, Y., Godwin, B., Conover, D., Kalbfleisch, T., Vijayadamodar, G., Yang, M., Johnston, M., Fields, S., and Rothberg, J. M. (2000) A comprehensive analysis of protein—protein interactions in Saccharomyces cerevisiae. Nature 403, 623-627.

1. A method for detecting an interaction between a polypeptide antigen and a single chain antibody variable fragment, comprising:
(a) culturing a population of host cells which contain:
a first reporter gene which expresses a first reporter protein when the first reporter gene is activated by a polypeptide which includes a transcriptional activation domain when the transcriptional activation domain is brought into sufficient proximity to a first binding site on or adjacent to the first reporter gene;
a second reporter gene which expresses a second reporter protein when the second reporter gene is activated by a polypeptide which includes a transcriptional activation domain when the transcriptional activation domain is brought into sufficient proximity to a second binding site on or adjacent to the second reporter gene;
a first hybrid protein comprising:
a first DNA-binding domain that recognizes the first binding site on or adjacent to the first reporter gene;
a first polypeptide antigen;
a second hybrid protein comprising:
the transcriptional activation domain;
a single chain antibody variable fragment;
a third hybrid protein comprising:
a second DNA-binding domain that recognizes the second binding site on or adjacent to a second reporter gene;
a second polypeptide antigen;
wherein interaction between the first polypeptide antigen and the single chain antibody variable fragment in the host cell causes the transcription activation domain to activate transcription of the first reporter gene;
wherein interaction between the second polypeptide antigen and the single chain antibody variable fragment in the host cell causes the transcription activation domain to activate transcription of the second reporter gene;
wherein cells in the population are homogeneous with respect to the polypeptide antigen and heterogeneous with respect to the single chain antibody variable fragment;
(b) selecting cells which (i) express the first reporter gene but not the second reporter gene, or (ii) which express the first and the second reporter genes, wherein expression of the first reporter gene is indicative of an interaction between the first polypeptide antigen and the single chain antibody variable fragment in a cell and expression of the second reporter gene is indicative of an interaction between the second polypeptide antigen and the single chain antibody variable fragment in a cell.

2. The method of claim 1 wherein the host cells further comprise:
a fourth hybrid protein comprising:
the second DNA-binding domain that recognizes the second binding site on or adjacent to the second reporter gene;
a third polypeptide antigen.

3. The method of claim 1 wherein the third hybrid protein further comprises:
at least a third polypeptide antigen.

4. The method of claim 1 wherein the host cells further comprise:
a fourth hybrid protein comprising:
a third DNA-binding domain that recognizes a third binding site on or adjacent to a third reporter gene;
a third polypeptide antigen; and
a third reporter gene which expresses a third reporter protein when the third reporter gene is activated by a polypeptide which includes a transcriptional activation domain when the transcriptional activation domain is brought into sufficient proximity to a binding site on or adjacent to the third reporter gene.

5. The method of claim 1 wherein the first and second polypeptide antigens are members of a protein family and share a greater than random degree of homology.

6. (canceled)

7. (canceled)

8. (canceled)

9. (canceled)

10. The method of claim 1 wherein the first reporter protein permits growth of the host cells under conditions which are otherwise non-permissive.

11. The method of claim 1 wherein the second polypeptide antigen comprises an inactivated form of the first DNA binding domain, wherein the inactivated form does not efficiently recognize the binding site on or adjacent to the first reporter gene.

12. (canceled)

13. The method of claim 1 wherein the first reporter protein can be assayed to determine amount of the first protein expressed in the cell.

14. (canceled)

15. (canceled)

16. The method of claim 1 wherein the second reporter protein creates a toxic product.

17. The method of claim 1 wherein expression of the first and second reporter proteins is determined simultaneously.

18. The method of claim 1 wherein expression of the first reporter protein is determined prior to determining expression of the second reporter protein.

19. (canceled)

20. (canceled)

21. (canceled)

22. (canceled)

23. A method for detecting an interaction between a polypeptide antigen and a single chain antibody variable fragment, comprising:
(a) culturing a population of first host cells which contain:
a first reporter gene which expresses a first reporter protein when the first reporter gene is activated by a polypeptide which includes a transcriptional activation domain when the transcriptional activation domain is brought into sufficient proximity to a binding site on or adjacent to the first reporter gene;
a first hybrid protein comprising:
a first DNA-binding domain that recognizes the binding site on or adjacent to the first reporter gene;
a first polypeptide antigen;
a second hybrid protein comprising:
the transcriptional activation domain;
a single chain antibody variable fragment;
(b) transferring a DNA molecule encoding the second hybrid protein from a cell which expresses the first reporter gene to a second host cell which comprises:
a second reporter gene which expresses a second reporter protein when the second reporter gene is activated by a polypeptide which includes a transcriptional activation domain when the transcriptional activation domain is brought into sufficient proximity to a second binding site on or adjacent to the second reporter gene;
a third hybrid protein comprising:
a second DNA-binding domain that recognizes the second binding site on or adjacent to a second reporter gene;
a second polypeptide antigen;
wherein interaction between the first polypeptide antigen and the single chain antibody variable fragment in the first host cell causes the first transcription activation domain to activate transcription of the first reporter gene;
wherein interaction between the second polypeptide antigen and the single chain antibody variable fragment in the second host cell causes the second transcription activation domain to activate transcription of the second reporter gene;
wherein cells in the population of first host cells are homogeneous with respect to the first polypeptide antigen and heterogeneous with respect to the single chain antibody variable fragment;
(c) selecting a second host cell which comprises the DNA molecule encoding the second hybrid protein but which (i) does not express the second reporter gene in the second host cell, or (ii) which does express the second reporter gene in the second host cell, wherein expression of the first reporter gene in a cell in the first population of host cells is indicative of an interaction between the first polypeptide antigen and the single chain antibody variable fragment, and expression of the second reporter gene is indicative of an interaction between the second polypeptide antigen and the single chain antibody variable fragment in the second host cell.

24. (canceled)

25. (canceled)

26. (canceled)

27. (canceled)

28. (canceled)

29. (canceled)

30. (canceled)

31. (canceled)

32. A population of host cells which comprise:
a first reporter gene which expresses a first reporter protein when the first reporter gene is activated by a polypeptide which includes a transcriptional activation domain when the transcriptional activation domain is brought into sufficient proximity to a first binding site on or adjacent to the first reporter gene;
a second reporter gene which expresses a second reporter protein when the second reporter gene is activated by a polypeptide which includes a transcriptional activation domain when the transcriptional activation domain is brought into sufficient proximity to a second binding site on or adjacent to the second reporter gene;
a first hybrid protein comprising:
a first DNA-binding domain that recognizes the first binding site on or adjacent to the first reporter gene;
a first polypeptide antigen;
a second hybrid protein comprising:
the transcriptional activation domain;
a single chain antibody variable fragment;
a third hybrid protein comprising:
a second DNA-binding domain that recognizes a second binding site on or adjacent to a second reporter gene;
a second polypeptide antigen;
wherein interaction between the first polypeptide antigen and the single chain antibody variable fragment in the host cell causes the transcription activation domain to activate transcription of the first reporter gene;
wherein interaction between the second polypeptide antigen and the single chain antibody variable fragment in the host cell causes the transcription activation domain to activate transcription of the second reporter gene;
wherein cells in the population are homogeneous with respect to the polypeptide antigen and heterogeneous with respect to the single chain antibody variable fragment.

33. (canceled)

34. The method of claim 1 or population of host cells of claim 23 wherein the cells are selected from the group consisting of bacterial, mammalian, yeast, and insect cells.

35. (canceled)

36. (canceled)

37. (canceled)

38. (canceled)

39. (canceled)

40. (canceled)

41. (canceled)

42. (canceled)

43. A kit which comprises in a single or divided container:
a first reporter gene which expresses a first reporter protein when the first reporter gene is activated by a polypeptide which includes a transcriptional activation domain when the transcriptional activation domain is brought into sufficient proximity to a first binding site on or adjacent to the first reporter gene;
a second reporter gene which expresses a second reporter protein when the second reporter gene is activated by a polypeptide which includes a transcriptional activation domain when the transcriptional activation domain is brought into sufficient proximity to a second binding site on or adjacent to the second reporter gene;
a vector for making first hybrid protein comprising:
a first DNA-binding domain that recognizes the first binding site on or adjacent to the first reporter gene;
and an insertion site for sequence encoding a first polypeptide antigen;
a library of vectors encoding second hybrid proteins each of which comprises:
the transcriptional activation domain;
a single chain antibody variable fragment;
a vector for making a third hybrid protein comprising:
a second DNA-binding domain that recognizes a second binding site on or adjacent to a second reporter gene;
an insertion site for a sequence encoding a second polypeptide antigen;
wherein interaction between the first polypeptide antigen and a single chain antibody variable fragment in a host cell causes the transcription activation domain to activate transcription of the first reporter gene;
wherein interaction between the second polypeptide antigen and a single chain antibody variable fragment in the host cell causes the transcription activation domain to activate transcription of the second reporter gene.

44. (canceled)

45. (canceled)
